(19) United States
(12) Patent Application Publication
(10) Pub. No.: US 2024/0261253 A1
(43) Pub. Date: Aug. 8, 2024

(54) DAURICHROMENIC ACID AND IT'S USE IN MEDICINE

(71) Applicant: Phytotherapeutix Ltd., Chester Cheshire (GB)

(72) Inventor: George Colin Stott, Southport Merseyside (GB)

(21) Appl. No.: 18/568,757

(22) PCT Filed: Jun. 9, 2022
(86) PCT No.: PCT/IB2022/055372

§ 371 Date: Dec. 8, 2023

(30) Foreign Application Priority Data
Jun. 10, 2021 (GB) 2108345.6

(51) Int. Cl. A61K 31/353; A61P 31/14

(52) U.S. Cl. CPC A61K 31/353; A61P 31/14

(57) ABSTRACT

A compound of daurichromenic acid (DCA, 2-[(3E)-4,8-Dimethyl-3,7-nonadien-1-yl]-5-hydroxy-2,7-dimethyl-2H-chromene-6-carboxylic acid or (E)-2-(4,8-dimethylnona-3,7-dien-1-yl)-5-hydroxy-2,7-dimethyl-2H-chromene-6-carboxylic acid) and it's use in medicine is disclosed. The DCA, or salts and hydrates thereof, is used in targeting and/or treating beta coronavirus infections and disease caused thereby.

This invention relates to Daurichromenic acid (DCA, 2-[(3E)-4,8-Dimethyl-3,7-nonadien-1-yl]-5-hydroxy-2,7-dimethyl-2H-chromene-6-carboxylic acid or (E)-2-(4,8-dimethylnona-3,7-dien-1-yl)-5-hydroxy-2,7-dimethyl-2H-chromene-6-carboxylic acid) and it's use in medicine. More particularly, it relates to the use of DCA, or salts and hydrates thereof, in targeting and/or treating beta coronavirus infections and disease caused thereby.

### BACKGROUND

Chromenic acids are naturally occurring compounds found in many plant species, but particularly in the Rhododendron species—e.g. Rhododendron dauricum L. (Ericaceae), which is distributed in Northern China, Eastern Siberia and Hokkaido. Rhododendron dauricum L. is a folk medicine used in China as an expectorant and mucolytic in the treatment of acute and chronic bronchitis. In China, the dried leaves of the plant are known as “Manshanfong”. The essential oil Manshanhongyou, Oleum Rhododendri daurici, is obtained by steam distillation of the dry leaves of R. dauricum. Its galenic preparation Manshanhongyou Jiaowan, Capsulae Olei Rhododendri daurici, is officially listed in the Chinese Pharmacopoeia. The oil is taken orally, 0.05-0.1 g, 2-3 times/day.

A methanolic extract of the leaves and twigs of R. dauricum has been found to possess significant anti-HIV activity in vitro (EC₅₀≤20 g/mL) [Kasiwada et al. 2001]. Subsequent analysis of some of the components present in the plant also demonstrated anti-viral activity against HIV. One of the components of R. dauricum, daurichromenic acid ((+)-DCA, (S,E)-2-(4,8-dimethylnona-3,7-dien-1-yl)-5-hydroxy-2,7-dimethyl-2H-chromene-6-carboxylic acid) was reported to have potent anti-HIV activity in vitro in acutely infected H9 cells with an EC₅₀ value of 5.67 ng/mL. It also inhibited uninfected H9 growth with an EC₅₀ value of 21.1 μg/mL, and thus, showed a good therapeutic index value of 3,710. Rhododaurichromanic acid A also showed relatively potent anti-HIV activity with EC₅₀ and Therapeutic Index (TI) value or 0.37 μg/mL and 91.9 μg/mL, respectively, whereas Rhododaurichromanic acid B displayed no anti-HIV activity [Kasiwada et al. 2001].

Given that Daurichromenic acid ((+)-DCA) demonstrated such potent anti-HIV activity in vitro, and the traditional use of R. dauricum L. is for the treatment of acute and chronic bronchitis, it was postulated that it may have other anti-viral activity, including anti-viral activity versus coronaviruses.

DCA is a chromenic acid compound comprising:
i) a chromene skeleton (A),
ii) an acid group moiety (B),
iii) a methyl group, attached to the benzene moiety of the chromene skeleton (C); and
iv) an unsaturated side chain (D).

Its structure is illustrated in FIG. 1.

The molecule is classed as an “orcinoid”, as it has an orcinol side chain (i.e. a methyl group). It is an analogue of Cannabichromenic Acid (CBCA), which is present in varying amounts in Cannabis sativa L, depending upon the strain of cannabis. It's structure is illustrated in FIG. 2.

There are two main differences in chemical structure between e.g. (+)-DCA and (+)-CBCA. These are that:
(i) CBCA has a pentyl group attached to the benzene ring of the chromene moiety (at the 3 position), and
(ii) (+)-DCA has a longer, more unsaturated side chain on the other side of the molecule (at the 3′ position)

Applicant speculated that orcinoid-like chromenic acids, which they define as a chromene or chromenic acid, with a methyl group in the 3 position, might have further interesting pharmacological and pharmaceutical activity.

Applicant also speculated that given that the chemical structure of DCA was similar to the cannabinoid, cannabichromenic acid (CBCA) and cannabichromevarinic acid (CBCVA), that DCA and other longer chain DCA analogues (e.g. diterpenodaurichromenic acid (DTDCA), where there is a longer unsaturated side chain in the 3′ position, might also have interesting pharmacological and pharmaceutical activity.

Applicant further speculated that given that the chemical structure of DCA was somewhat similar to the cannabinoid cannabichromenic acid (CBCA) and cannabichromevarinic acid (CBCVA), which, like their corresponding decarboxylated derivatives cannabichromene (CBC) and cannabichromevarin (CBCV), has good safety and tolerability in animals and humans, that DCA and other longer chain DCA analogues e.g. diterpenodaurichromenic acid (DTDCA) and their decarboxylated derivatives could have similar good safety and tolerability in humans and animals.

Additionally, given that the chemical structure of DCA was somewhat similar to the cannabinoid cannabichromenic acid (CBCA), which, does not easily penetrate the blood-brain-barrier (BBB) in mammals, they speculated that DCA and other longer chain DCA analogues (e.g. diterpenodaurichromenic acid (DTDCA) might have similar pharmacokinetic profiles in mammals, and not readily cross the BBB. The structure of DTDCA is illustrated in FIG. 3. Hence, it is unlikely that DCA and DTDCA (and analogues thereof) might penetrate the BBB in mammals and produce central or psychoactive/psychotropic effects.

An alternative chemical name for (+) daurichromenic acid is: (2S)-2-[(3E)-4,8-dimethylnona-3,7-dienyl]-5-hydroxy-2,7-dimethylchromene-6-carboxylic acid (IUPAC Name)

An alternative name for (+) cannabichromenic Acid (CBCA) is: 5-hydroxy-2-methyl-2-(4-methylpent-3-enyl)-7-pentylchromene-6-carboxylic acid (IUPAC Name)

An alternative chemical name for (+) diterpenodaurichromenic acid is: (2S)-2-[(3E)-4,8,12-trimethyltrideca-3,7,11-trienyl]-5-hydroxy-2,7-dimethylchromene-6-carboxylic acid (IUPAC Name).

The biosynthesis of DCA and CBCA are described in Taura et al. 2018 (Daurichromenic acid and grifolic acid: Phytotoxic meroterpenoids that induce cell death in cell culture of their producer Rhododendron dauricum) and lijima et al. 2017 (Identification and Characterization of Daurichromenic Acid Synthase Active in Anti-HIV Biosynthesis).

The synthesis of a racemate of (±)-DCA has been described by Lee and Wang (2005).

Identified prior art include:
CN104415083;
JP201214462;
CN110604759;
Tetrahydron, vol 57, 2001; and
WO2004/058738.

It is an object of the present invention to identify further medical uses for DCA and by extrapolation consider the use of analogous compounds such as CBCA and DTDCA.

### SUMMARY

In accordance with a first aspect of the present invention there is provided a compound, daurichromenic acid (DCA) or a salt or hydrate thereof, for use in the treatment or prevention of disease caused by a beta coronavirus.

In a preferred embodiment the compound is used to treat the beta coronavirus disease 19 (SARS-CoV-2, known as “COVID 19”) or variants thereof. It may also be used to treat Middle East Respiratory Syndrome (MERS) and Severe Acute Respiratory Syndrome (SARS).

In one embodiment the compound is the isomeric form: (+)-(2S)-2-[(3E)-4,8-dimethylnona-3,7-dienyl]-5-hydroxy-2,7-dimethylchromene-6-carboxylic acid [(+)-DCA] or a salt or hydrate thereof.

In another embodiment the compound is the isomeric form: (−)-(2S)-2-[(3E)-4,8-dimethylnona-3,7-dienyl]-5-hydroxy-2,7-dimethylchromene-6-carboxylic acid [(−)-DCA] or a salt or hydrate thereof.

In yet another embodiment the compound is a racemic mixture of both the (+) and (−) forms, or a salt or hydrate thereof.

The racemic mixture can be in any ratio but more typically is in a ratio of from 1:2 to 2:1, most typically substantially 1:1.

The compounds, whatever their form, are of a pharmaceutical grade. They thus have a purity of at least 95%, more preferably still at least 98%, and most preferably still at least 99%.

In accordance with a second aspect of the present invention there is provided a pharmaceutical composition comprising daurichromenic acid (DCA) or a salt or hydrate thereof, together with at least one excipient and wherein the DCA or a salt or hydrate thereof is present in an amount providing a human unit dose of at least 0.5 mg/Kg.

For an adult weighing 70 Kg this would equate to a dose of 35 mg.

The daurichromenic acid or a salt or hydrate thereof may be the isomer (+)-DCA or the isomer (−)-DCA or a racemic mix of the two.

The composition may be formulated for delivery by any standard pharmaceutical route including parenteral, (intraperitoneal (i.p.), intravenous (i.v.), intramuscular (i.m.), and subcutaneous (s.c.)), oral, inhaled, nasal including nasogastric, ocular, transmucosal (buccal, vaginal, and rectal), intravesical, and transdermal.

The identified active pharmaceutical agents are particularly suited to treat the infection, or conditions caused by the infection, of beta-coronaviruses (including SARS-CoV-2, COVID-19). This is based on the results obtained in two well understood in vitro anti-viral models, using an alpha-coronavirus (E229 strain) and beta-coronavirus (OC-43 strain).

The anti-viral agent, Remdesivir, was used as a positive control in these in vitro experiments.

Based on this early data one might anticipate a human i.p. dose to be in the order of from 0.5 mg/Kg or as a daily i.p. or oral dose for a (70 Kg) adult patient from 28 mg DCA.

In accordance with a third aspect of the present invention there is provided a method of treating a subject comprising administering to the patient an effective amount of (+)-DCA, (−)-DCA or a 1:1 ratio of (+)-DCA:(−)-DCA in a unit dosage form.

The patient may be an adult, child, neonate or infant.

In one embodiment the dose is in the form of a parenteral (intraperitoneal, intravenous, intramuscular, and subcutaneous), oral, inhaled, nasal including nasogastric, ocular, transmucosal (buccal, vaginal, and rectal), intravesical, or transdermal form.

Preferably the method of treatment or prevention is as an anti-viral, more particularly an anti-beta-coronavirus agent.

### BRIEF DESCRIPTION OF THE DRAWINGS

An embodiment of the invention is further described hereinafter with reference to the accompanying drawing, in which:

FIG. 1a illustrates the (non-stereo-specific) structure of daurichromenic acid (DCA);

FIG. 1b illustrates the structure of the (+) isomer of daurichromenic acid ((+) DCA);

FIG. 1c illustrates the structure of the (−) isomer of daurichromenic acid ((−)-DCA);

FIG. 2a illustrates the (non-stereo-specific) structure of Cannabichromenic acid (CBCA);

FIG. 2b illustrates the structure of the (+) isomer of Cannabichromenic acid ((+)-CBCA);

FIG. 2c illustrates the structure of the (−) isomer of Cannabichromenic acid ((−)-CBCA);

FIG. 3a illustrates the (non-stereo-specific) structure of diterpenodaurichromenic acid (DTDCA);

FIG. 3b illustrates the structure of the (+) isomer of diterpenodaurichromenic acid ((+)-DTDCA);

FIG. 3c illustrates the structure of the (−) isomer of diterpenodaurichromenic acid ((−)-DTDCA);

FIG. 4a is a scheme for the manufacture of DCA;

FIG. 4b shows step 1 in more detail;

FIG. 4c shows step 2 in more detail;

FIG. 4d shows step 3 in more detail;

FIG. 4e shows a literature identification of a racemate and the (+) isomer of the methyl ester salt of DCA;

FIG. 4f shows the identification of a racemate and the (+) isomer of an exemplary salt (the methyl ester) produced;

FIG. 5 shows the cytotoxicity of DCA in an alpha coronavirus model. It shows % viability of 16HBE cells plotted against increasing concentrations of the compounds. Data are presented as mean percentage cell viability ±SEM (n=3);

FIG. 6 shows the efficacy of DCA in an alpha coronavirus model. It shows % inhibition of HCoV 229E infection in 16HBE cells plotted against increasing concentrations of the compounds. Data are presented as mean percentage inhibition of viral infection ±SEM (n=3);

FIG. 7 shows the % inhibition of HCoV 229E infection in 16HBE cells plotted against increasing concentrations of remdesivir. Data are presented as mean percentage inhibition of viral infection ±SEM (n=3);

FIG. 8 shows the cytotoxicity of DCA in a beta coronavirus model. It shows % viability of H292 cells plotted against increasing concentrations of the compound. Data are presented as mean percentage cell viability ±SEM (n=3);

FIG. 9 shows the efficacy of DCA in a beta coronavirus model. It shows % inhibition of HCoV OC43 infection in H292 cells plotted against increasing concentrations of the compounds. Data are presented as mean percentage inhibition of viral infection ±SEM (n=3); and

FIG. 10 shows the efficacy of a positive control (Remdisivir) in an alpha coronavirus model. It shows % inhibition of HCoV OC43 infection in H292 cells plotted against increasing concentrations of remdesivir. Data are presented as mean percentage inhibition of viral infection ±SEM (n=3).

### DETAILED DESCRIPTION

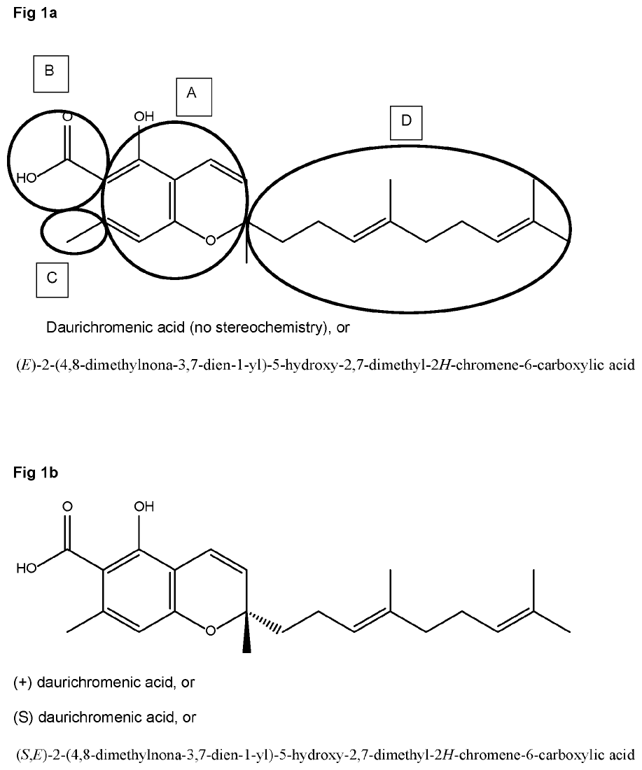

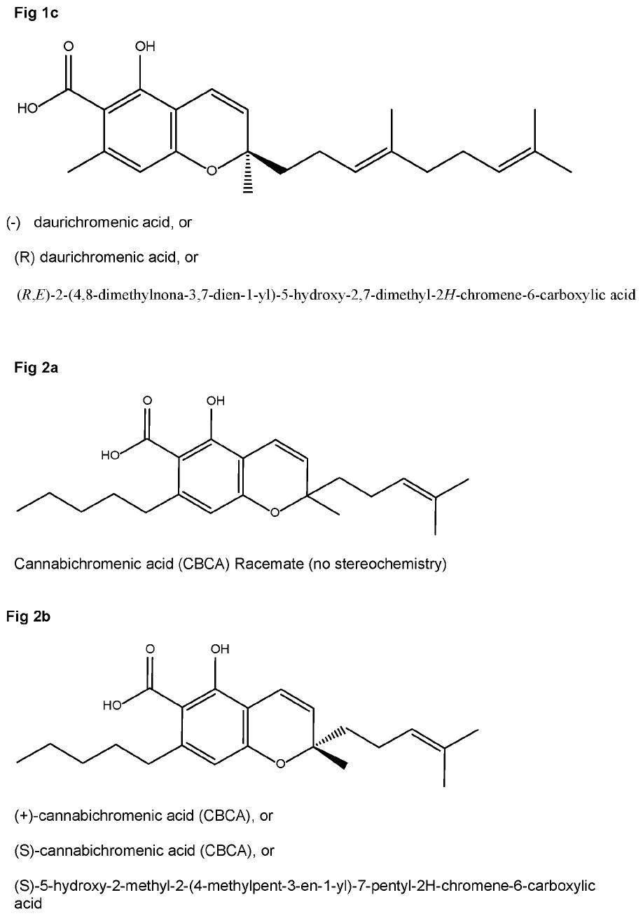

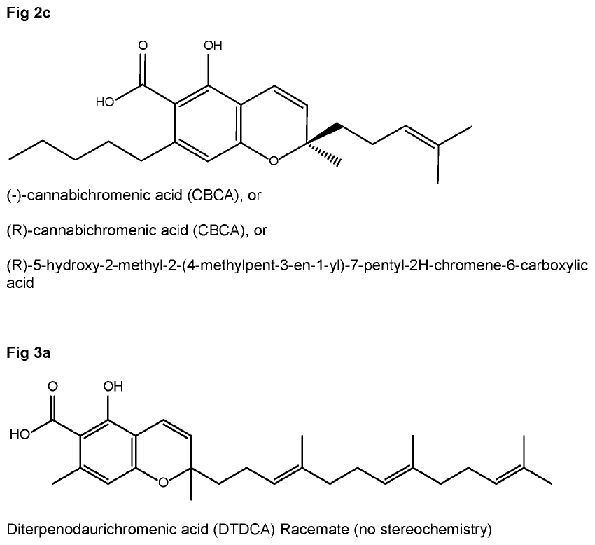

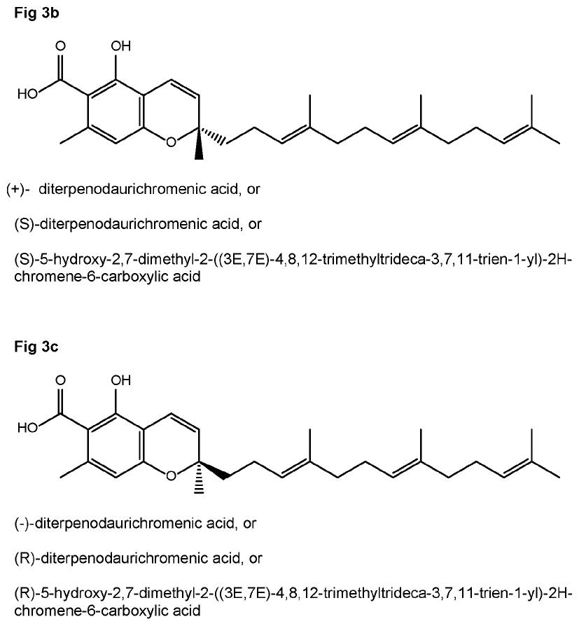

The first challenge was to make the compound DCA (FIG. 1), and more particularly separate the stereo-specific (+) and (−) isomers (FIG. 1b and FIG. 1c respectively) from the racemic mixture.

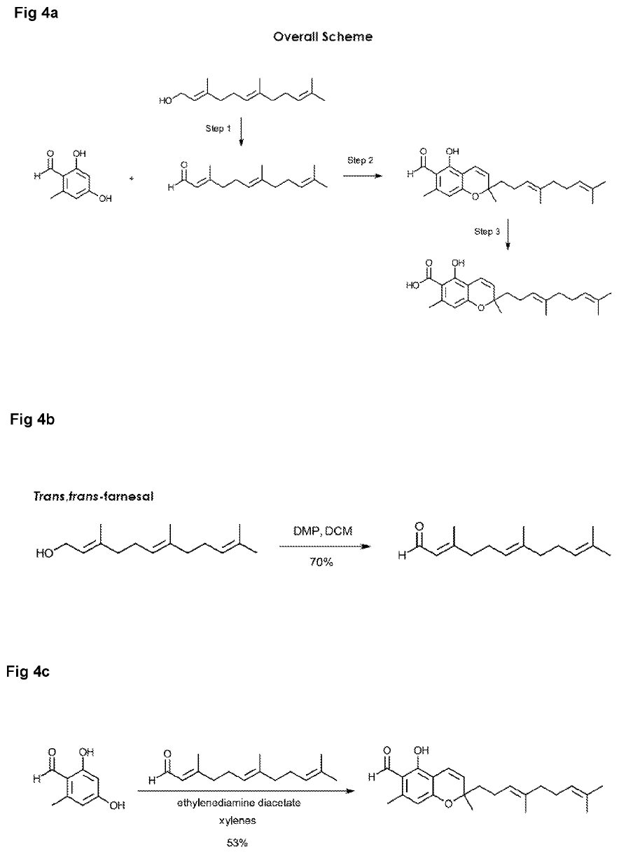

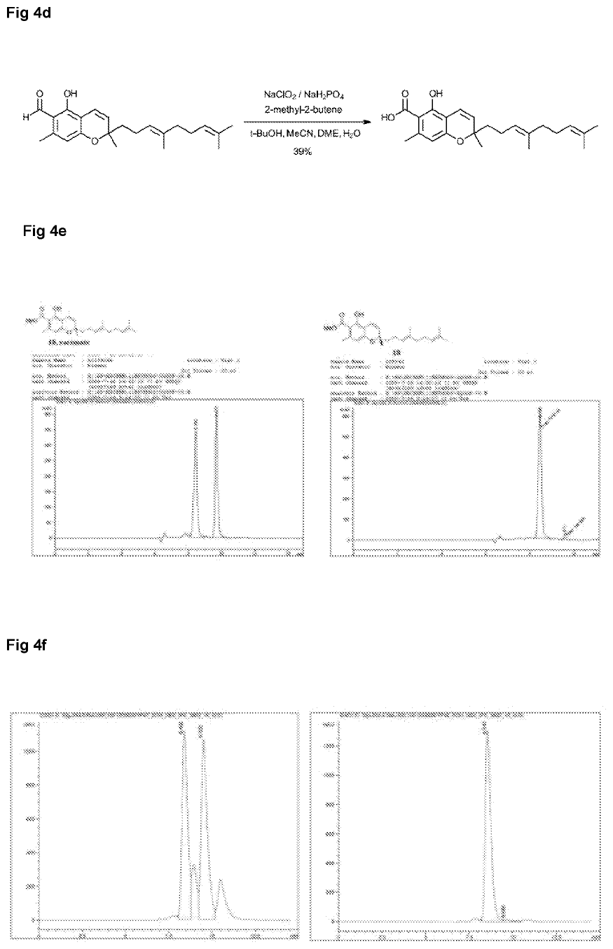

The compounds of the invention were produced by way of the general scheme illustrated in FIG. 4a (and more particularly in FIGS. 4b, 4c and 4d).

Synthesis was carried out following literature conditions as set out under Methodology. Similar yields to those reported were obtained to deliver 1.1 g of the target racemic daurichromenic acid (DCA) at 93% purity by LCMS.

External analysis of the racemate found chiral chromatography conditions which gave good separation of the enantiomers. However, close running impurities not seen in the High Performance Liquid Chromatography (HPLC) method at Apex Molecular led to a decision that direct chiral purification was not practical. The DCA racemate was initially purified by asymmetric preparative HPLC to remove the impurities causing problems, followed by chiral preparative Supercritical Fluid Chromatography (SFC).

To confirm the identity of the enantiomers, a portion of racemic DCA and of one of the separated enantiomers were converted to the methyl esters. These were analysed under literature conditions allowing comparison to reported chiral DCA data and assignment of the separated enantiomers.

### Methodology

The general methodology is as set out in FIG. 4a.

### Step 1 (FIG. 4b)

Referring to FIG. 4b, to a solution of trans, trans-farnesol (5.0 g, 22.5 mmol) in dichloromethane (100 mL) was added Dess-Martin periodinane (12.4 g, 29.2 mmol, 1.3 equiv.). The reaction mixture was stirred at room temperature overnight. NaHCO₃ (sat. aq., 100 mL) was added. The organic layer was washed with NaHCO₃ (sat. aq., 100 mL), brine, dried (MgSO₄) and evaporated. The residue was purified by dry flash chromatography (5% ethyl acetate in petroleum ether) to give the desired product as a colourless oil (3.49 g, 70%).

The chemical structure was confirmed by 1H NMR—the details of which are as follows:

1H NMR (500 MHz, CDCl3) δ 9.99 (d, J=8.1 Hz, 1H), 5.88 (d, J=8.1 Hz, 1H), 5.07 (q, J=8.4, 7.6 Hz, 2H), 2.27-2.19 (m, 4H), 2.17 (s, 3H), 2.09-2.03 (m, 2H), 2.00-1.95 (m, 2H), 1.67 (s, 3H), 1.60 (s, 3H), 1.59 (s, 3H).

### Step 2 (FIG. 4c)

Referring to FIG. 4c, 2,4-Dihydroxy-6-methylbenzaldehyde (2.00 g, 13.2 mmol) and trans, trans-farnesal (3.48 g, 15.8 mmol, 1.2 equiv.) were dissolved in xylene (100 mL), and ethylenediamine diacetate (EDTA, 238 mg, 1.32 mmol, 0.1 equiv.) was added at room temperature. The mixture was heated at reflux temperature for 6 h. The solvent was evaporated, and the residue purified by column chromatography (5% ethyl acetate in petroleum ether) to give the desired product as a yellow oil (2.48 g, 53%).

The chemical structure was confirmed by ¹H (proton) NMR—the details of which are as follows:

¹H NMR (500 MHz, CDCl3) δ 12.65 (s, 1H), 10.0 (s, 1H), 6.69 (d, J=10.1 Hz, 1H), 6.17 (s, 1H), 5.49 (d, J=10.1 Hz, 1H), 5.13-5.04 (m, 2H), 2.48 (s, 3H), 2.13-2.00 (m, 4H), 1.98-1.92 (m, 2H), 1.81-1.73 (m, 1H), 1.67 (s, 3H), 1.67-1.59 (m, 1H), 1.59 (s, 3H), 1.56 (s, 3H), 1.41 (s, 3H).

UPLC-LCMS (Method A): 97% @ 254 nm, RT=3.20 min, ES+355.3=[M+H]+.

### Step 3 (FIG. 4d)

To a solution of 5-hydroxy-2,7-dimethyl-2-(4,8-dimethyl-3E,7-nonadienyl)-2H-chromene-6-carbaldehyde (2.48 g, 7.00 mmol) in t-butanol (38 mL), acetonitrile (38 mL), 2-methyl-2-butene (25 mL), and DME (13 mL) was added NaH₂PO₄ (4.2 g, 35.0 mmol) and NaClO₂ (3.17 g, 35.0 mmol, dissolved in 13 mL of water) at 0° C. The resulting mixture was warmed slowly to room temperature and stirred for 36 h. LCMS showed the reaction to be incomplete so, after cooling back to 0° C., further NaH₂PO₄ (2.1 g, 17.5 mmol) and NaClO₂ (1.58 g, 17.5 mmol, dissolved in 7 mL of water) were added. After 18 hours the reaction was terminated, despite incomplete conversion. Brine (350 mL) was added, and the resultant solution was extracted with ethyl acetate (3×350 mL). The combined organic extracts were washed with brine (500 mL), dried over anhydrous MgSO₄ and evaporated. The residue was purified by flash chromatography (4:1→3:1 petroleum ether:ethyl acetate to give the desired product as an orange oil (1.0 g, 39%).

The chemical structure was confirmed by ¹H NMR—the details of which are as follows:

¹H NMR (500 MHz, CDCl3) δ 11.75 (s, 1H), 6.73 (d, J=10.1 Hz, 1H), 6.23 (s, 1H), 5.48 (d, J=10.1 Hz, 1H), 5.13-5.05 (m, 2H), 2.53 (s, 3H), 2.15-1.90 (m, 6H), 1.82-1.71 (m, 2H), 1.67 (s, 3H), 1.59 (s, 3H), 1.57 (s, 3H), 1.40 (s, 3H).

LCMS (Method B): 93% @ 254 nm, RT=2.30 min, ES-369.2=[M−H]−.

### Separation of Enantiomers

Separation was by chiral column. Purification was carried out by asymmetric preparative HPLC followed by chiral SFC.

### Asymmetric Preparative HPLC

The asymmetric preparative HPLC conditions were as follows:
Column Details Gemini NX C18 (150×50 mm, Sum)
Column Temperature Ambient
Flow Rate 118 mL/minute
Detector Wavelength 250 nm
Injection Volume 2000 uL (120 mg)
Mobile Phase A Water (0.2% v/v NH3)
Mobile Phase B MeCN
   Details are given in Table 1

TABLE 1
Gradient Profile

| Time(mins) | % A | % B |
|---|---|---|
| 0 | 80 | 20 |
| 1 | 80 | 20 |
| 1.1 | 75 | 25 |
| 8.5 | 48 | 52 |
| 8.6 | 0 | 100 |
| 10.5 | 0 | 100 |
| 10.6 | 80 | 20 |

### Chiral Preparative Supercritical Fluid Chromatography (SFC)

The preparative SFC conditions were as follows:
Column Details Chiralpak IG (20 mm×250 mm, 5 um) Column Temperature 40° C.
Flow Rate 50 mL/min
BPR 125 BarG
Detector Wavelength 251 nm
Injection Volume 300 uL (6 mg)
Isocratic Conditions 30:70 MeOH:CO₂

### Chiral Purity Analysis

The analysis was as follows:
Chiral Purity Analysis Conditions;
Column Details Chiralpak IG (4.6 mm×250 mm, 5 um) Column Temperature 40° C.
Flow Rate 4 mL/min
Detector Wavelength 210-400 nm
Injection Volume 1.0 uL
BPR 125 BarG
Isocratic Conditions 20:80 MeOH:CO₂

### Isolated Products

YP44_1_1 (−) daurichromenic Acid
Chemical purity 95.47%, m/z=371.11
Enantiomeric excess 98.6
   YP44_1_2 (+) daurichromenic Acid
Chemical purity 100.00%, m/z=371.11
Enantiomeric excess 99.4

### Synthesis of Methyl Ester Salt.

Literature conditions (Liu and Woggon, EUR. J. Chem., 2010, 1033) were used to convert racemic (±)-DCA and (+) DCA to their methyl esters using TMS diazomethane.

### Identification of Enantiomers

Literature chiral HPLC data for DCA methyl ester racemate (FIG. 4e) and (+)-DCA methyl ester racemate (FIG. 4e) as given in Liu and Woggon (above) were used to compare the isolated products obtained based on equivalent reaction times and order of elution (FIG. 4f) which confirmed their production and separation.

### Anti-Viral Assay—Experimental Conditions:

To investigate the effect of DCA, its enantiomers, and analogues the racemic mixture of DCA and its' two enantiomers were tested against two representative coronavirus, an alpha (229E) and a beta (OC43) coronavirus.

A dilution series of each test compound (8-point, 10-fold dose titration) was added prophylactically for one hour, to cells. Cells were infected with virus for one hour, at a single concentration (100×TCID₅₀ (Median Tissue Culture Infectious Dose). Additional media was added to the wells, with equivalent concentrations of compound added in for the duration of the study. Vehicle and positive control wells were set up to control for any influence of the compounds alone on cell viability. Cells were visually inspected for the appearance of any cytopathic effects (CPE). A cell viability assay was performed once CPE was complete.

Inhibition was calculated as follows:

$$\% Inhibition = [ ( A - B ) / ( C - B ) ] \times 100 ,$$

where:
A: mean optical density of test,
B: mean optical density of virus controls,
C: mean optical density of cell controls.
Negative values occur when A<B, due either to natural variation or compound toxicity.

### Readouts:

EC₅₀: The concentration which results in 50% viral inhibition, following the addition of compounds.
CC₅₀: The concentration which results in 50% cell viability, following the addition of compounds.

### Assay Conditions

The assay conditions are given in Table 2 below:

TABLE 2

| Virus | Strain | Cell Type | Assay Duration |
|---|---|---|---|
| Coronavirus (HCoV) | 229E | 16HBE | 5 days |
| Coronavirus (HCoV) | OC43 | NCI-H292 | 5 days |

The compounds used, and their concentration are given in Table 3 below:

TABLE 3

| Compound | Concentration range |
|---|---|
| (+)-daurichromenic acid (DCA) | 100 μg/ml-0.01 ng/ml |
| (−)-daurichromenic acid (DCA) | 100 μg/ml-0.01 ng/ml |
| 1:1 mix of (+)-DCA and (−)-DCA | 200 μg/ml-0.02 ng/ml |
| Remdesivir (positive control) | 20 M-0.002 nM |

### Methodology

1. Cells permissible to infection, indicated in the Conditions Table, were grown and seeded into 96-well plates to a confluency of 80-90%.
2. The test compounds were serially diluted 10-fold into 8 concentrations in total. The serial dilution was added to the cells for 1 hour prior to infection with each virus. Positive control compounds were included using the same treatment regimen.
3. After pre-treatment, each virus was added to the cells for 1 h at 100×TCID₅₀. A mock infection of blank media was added to the uninfected controls.
4. After infection, the virus/media was removed and an overlay medium was added, with equivalent concentrations of the test compounds added in.
5. Cells were incubated until extensive cytopathic effects (CPE) were seen in the infected control wells.
6. A cell viability assay was performed on all conditions, with cells receiving the test treatment compared to the vehicle treated and uninfected controls.

### Results

### Human Coronavirus 229E

### Cytotoxicity

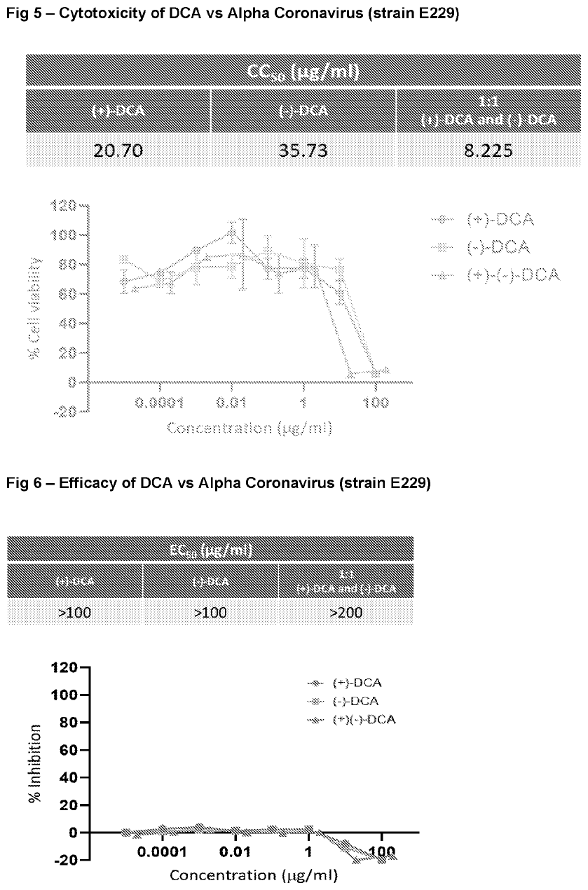

As illustrated in FIG. 5, which shows % viability of 16HBE cells plotted against increasing concentrations of the compound, the compounds are toxic (CC₅₀) at concentrations ranging from 8.2 μg/ml for a 1:1 racemic mixture to 35.73 for the (−) enantiomer. Data are presented as mean percentage cell viability ±SEM (n=3).

### Efficacy

As illustrated in FIG. 6, which shows % inhibition of HCoV 229E infection in 16HBE cells plotted against increasing concentrations of the compound, the compounds lacked efficacy. Data are presented as mean percentage inhibition of viral infection ±SEM (n=3).

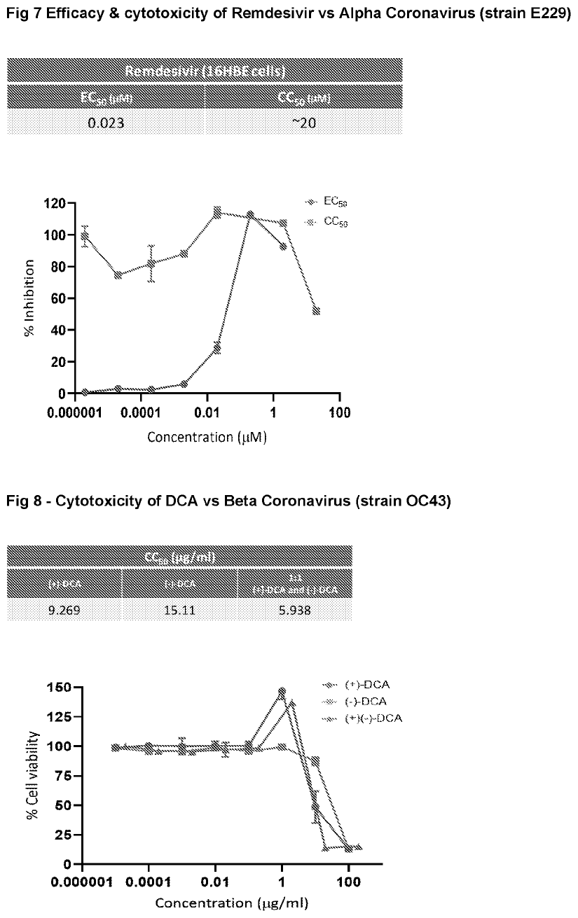

In contrast, as illustrated in FIG. 7, the positive control Remdesivir was highly effective with an EC₅₀ at 0.023 μg/ml when tested under the same conditions.

### Human Coronavirus OC43

### Cytotoxicity

As illustrated in FIG. 8, which shows % viability of H292 cells plotted against increasing concentrations of the compound, the compounds are toxic (CC₅₀) at concentrations ranging from 5.9 μg/ml for a 1:1 racemic mixture to 15.11 for the (−) enantiomer. Data are presented as mean percentage cell viability±SEM (n=3).

### Efficacy

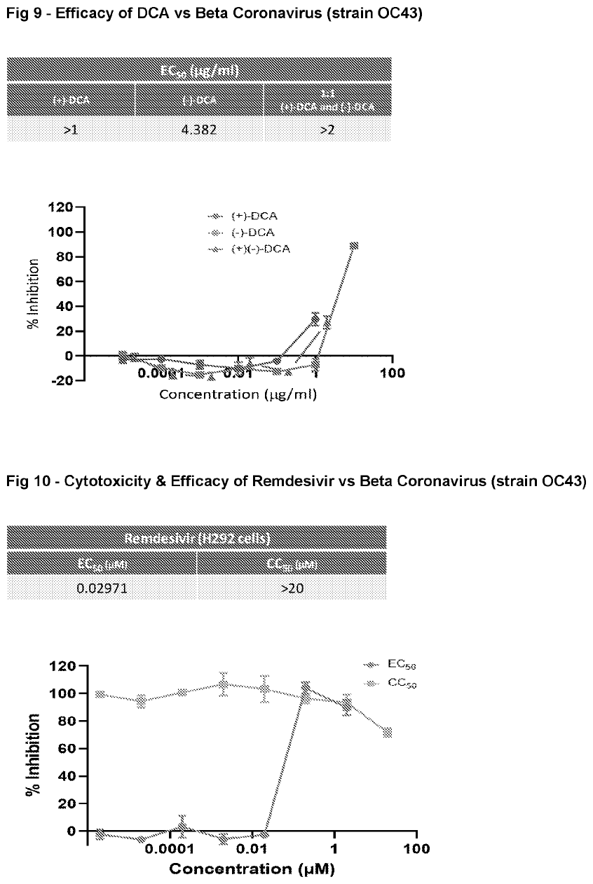

As illustrated in FIG. 9, which shows % inhibition of HCoV 043 infection in H292 cells plotted against increasing concentrations of the compound, the compounds exhibited efficacy, with (−) DCA having an EC₅₀ of 4.389 μg/ml. Data are presented as mean percentage inhibition of viral infection ±SEM (n=3).

As illustrated in FIG. 10, the positive control Remdesivir was highly effective with an EC₅₀ at 0.029 μg/ml when tested under the same conditions, several orders of magnitude higher.

However, given the structural similarity with CBCA and its extremely favourable toxicology profile this difference in dose is not expected to be problematic.

### Conclusions

The expected level of infection was achieved by all viruses in each assay.

Remdesivir worked as expected, with EC₅₀ values between 10 nM and 100 nM against both OC43 and 229E.

### Human Coronavirus 229E (Alpha):

(−)-DCA showed marginally less toxicity than (+)-DCA in 16HBE cells, with CC₅₀ values of 35.73 μg/ml and 20.70 μg/ml, respectively.

A 1:1 mixture of (+)-DCA with (−)-DCA produced higher toxicity than (+)-DCA or (−)-DCA alone, with a CC₅₀ value of 8.225 μg/ml.

No activity of any test compound was observed against HCoV 229E, with EC₅₀ values predicted to be substantially higher than the top concentration tested.

### Human Coronavirus OC43 (Beta):

(−)-DCA showed less toxicity than (+)-DCA in H292 cells, with CC₅₀ values of 15.11 μg/ml and 9.269 μg/ml, respectively.

A 1:1 mixture of (+)-DCA with (−)-DCA produced toxicity marginally lower than (+)-DCA alone, with a CC₅₀ value of 5.938 μg/ml. This is surprising given it is effectively twice the dose.

Efficacy was seen with (+)-DCA, (−)-DCA and a 1:1 mix of both against HCoV OC43.

(−)-DCA was tolerated at higher concentrations than (+)-DCA, it was possible to calculate an EC₅₀ value for (−)-DCA against HCoV OC43 (4.382 μg/m).

A 1:1 mixture of (+)-DCA with (−)-DCA produced similar viral inhibition to (+)-DCA alone.

Given that the EC50 for (−)-DCA was calculated to be 4.382 μg/mL, it is possible that this isomer could be the most potent of the 3 agents.

It is interesting to note that a 5 mg/kg i.p. dose of CBCA in rodents achieves a plasma Cmax of approx. 4 μg/mL, without any major side effects (Anderson et al. 2020, Anderson et al. 2021). Hence one would anticipate, given the chemical structural similarity between CBCA and DCA that DCA would be as well tolerated as CBCA, and that such therapeutic levels could be tolerated and achieved in vivo.

1. A compound, daurichromenic acid, or a salt or hydrate thereof, for use in the treatment or prevention of a disease caused by a beta coronavirus.

2. The compound, daurichromenic acid, or a salt or hydrate thereof, for use as claimed in claim 1, wherein the compound is used to treat diseases caused by beta-coronaviruses (including SARS-CoV-2, COVID-19) or variants thereof.

3. The compound, daurichromenic acid, or a salt or hydrate thereof, for use as claimed in claim 1, wherein the compound is used to treat Middle East Respiratory Syndrome (MERS) or Severe Acute Respiratory Syndrome (SARS).

4. The compound, daurichromenic acid, or a salt or hydrate thereof, for use as claimed in claim 1, wherein the compound is for use as an anti-beta coronavirus agent.

5. The compound, daurichromenic acid, or a salt or hydrate thereof, for use as claimed in claim 1, wherein the compound is one of:
i) (+)-(2S)-2-[(3E)-4,8-dimethylnona-3,7-dienyl]-5-hydroxy-2,7-dimethylchromene-6-carboxylic acid [(+)-DCA] or a salt or hydrate thereof; or
ii) (−)-(2R)-2-[(3E)-4,8-dimethylnona-3,7-dienyl]-5-hydroxy-2,7-dimethylchromene-6-carboxylic acid [(−)-DCA] or a salt or hydrate thereof, or
iii) a racemic mixture of both i) [(+)-DCA] and ii) [(−)-DCA], or a salt or hydrate thereof.

6. The compound, daurichromenic acid, or a salt or hydrate thereof, for use as claimed in claim 5, which is (+)-(2S)-2-[(3E)-4,8-dimethylnona-3,7-dienyl]-5-hydroxy-2,7-dimethylchromene-6-carboxylic acid [(+)-DCA] or a salt or hydrate thereof.

7. The compound, daurichromenic acid, or a salt or hydrate thereof, for use as claimed in claim 5, which is (−)-(2R)-2-[(3E)-4,8-dimethylnona-3,7-dienyl]-5-hydroxy-2,7-dimethylchromene-6-carboxylic acid [(−)-DCA] or a salt or hydrate thereof.

8. The compound, daurichromenic acid, or a salt or hydrate thereof, for use as claimed in claim 6, which is present as a substantially pure enantiomer.

9. The compound, daurichromenic acid, or a salt or hydrate thereof, for use as claimed in claim 8, wherein the substantially pure enantiomer is present at greater than 95% purity.

10. The compound, daurichromenic acid, or a salt or hydrate thereof, for use as claimed in claim 5, which is a racemic mixture of both i) [(+)-DCA] and ii) [(−)-DCA], or a salt or hydrate thereof.

11. The compound, daurichromenic acid, or a salt or hydrate thereof, for use as claimed in claim 10, wherein the compounds both i) [(+)-DCA] and ii) [(−)-DCA], or a salt or hydrate thereof are present in a ratio of from 1:2 to 2:1.

12. The compound, daurichromenic acid, or a salt or hydrate thereof, for use as claimed in claim 1, which is packaged for delivery by one of the following routes: parenteral, oral, inhaled, nasal including nasogastric, ocular, transmucosal or transdermal.

13. The compound, daurichromenic acid, or a salt or hydrate thereof, for use as claimed claim 1, which is provided at a dose of at least 0.5 mg/Kg.

14. A pharmaceutical composition, comprising: daurichromenic acid (DCA) or a salt or hydrate thereof, together with at least one excipient, and wherein the DCA or a salt or hydrate thereof is present in an amount providing a human unit dose of at least 0.5 mg/Kg.

15. A method of treating a subject infected with a beta-coronavirus comprising: administering to the patient an effective amount of daurichromenic acid, or a salt or hydrate thereof.

16. The method as claimed in claim 15, wherein the daurichromenic acid is for the treatment of SARS-CoV-2 (COVID-19).

17. The method as claimed in claim 15, wherein the daurichromenic acid, or a salt or hydrate thereof, is one of:
i) (+)-(2S)-2-[(3E)-4,8-dimethylnona-3,7-dienyl]-5-hydroxy-2,7-dimethylchromene-6-carboxylic acid [(+)-DCA] or a salt or hydrate thereof; or
ii) (−)-(2R)-2-[(3E)-4,8-dimethylnona-3,7-dienyl]-5-hydroxy-2,7-dimethylchromene-6-carboxylic acid [(−)-DCA] or a salt or hydrate thereof, or
iii) a racemic mixture of both i) [(+)-DCA] and ii) [(−)-DCA], or a salt or hydrate thereof.

18. The method as claimed in claim 17, wherein both i) [(+)-DCA] and ii) [(−)-DCA], or a salt or hydrate thereof are present in a ratio of from 1:2 to 2:1.

19. The pharmaceutical composition as claimed in claim 14, wherein the DCA or a salt or hydrate thereof is one of:
i) (+)-(2S)-2-[(3E)-4,8-dimethylnona-3,7-dienyl]-5-hydroxy-2,7-dimethylchromene-6-carboxylic acid [(+)-DCA] or a salt or hydrate thereof; or
ii) (−)-(2R)-2-[(3E)-4,8-dimethylnona-3,7-dienyl]-5-hydroxy-2,7-dimethylchromene-6-carboxylic acid [(−)-DCA] or a salt or hydrate thereof, or
iii) a racemic mixture of both i) [(+)-DCA] and ii) [(−)-DCA], or a salt or hydrate thereof.

20. The pharmaceutical composition as claimed in claim 19, wherein both i) [(+)-DCA] and ii) [(−)-DCA], or a salt or hydrate thereof are present in a ratio of from 1:2 to 2:1.
